(19) United States
(12) Patent Application Publication
(10) Pub. No.: US 2021/0082576 A1
(43) Pub. Date: Mar. 18, 2021

(54) PLANT AND ANIMAL HEALTH MONITORING MANAGEMENT SYSTEM AND ITS METHOD

(71) Applicant: Jiedong Zhong, Gaozhou (CN)

(72) Inventor: Jiedong Zhong, Gaozhou (CN)

(21) Appl. No.: 17/035,853

(22) Filed: Sep. 29, 2020

Related U.S. Application Data
(63) Continuation of application No. PCT/CN2019/105523, filed on Sep. 12, 2019.

(51) Int. Cl. G16H 50/20; A61B 5/0205; A01G 7/00; G16H 40/67; G16H 20/00; G16H 15/00

(52) U.S. Cl. CPC G16H 50/20; A61B 5/02055; A01G 7/00; A61B 5/024; G16H 20/00; G16H 15/00; G16H 40/67

(57) ABSTRACT

The present disclosure provides a plant and animal health monitoring management system and its method. The plant and animal health monitoring management system comprise a plant and animal health monitoring unit, an AI and artificial integrated health control unit, a big data monitoring management center unit, a plant and animal operation supervision unit, and a data analysis monitoring unit. The AI and artificial integrated health control unit comprises an AI diagnosis and treatment control system, an AI treatment control system, an artificial intervention analysis system and an artificial intervention execution unit.

### CROSS REFERENCE TO RELATED APPLICATIONS

The present application is a Continuation Application of PCT Application No. PCT/CN2019/105523 filed on Sep. 12, 2019, the contents of which are incorporated herein by reference in their entirety.

### TECHNICAL FIELD

Embodiments of the disclosure generally relate to monitoring management system and method, and more particularly to a plant and animal health monitoring management system and its method.

### BACKGROUND

At present, the supervision of animals and plants is mainly based on manual data collection and manual data processing. The workload of obtaining global data for animal management and maintenance is enormous. The data of disease and epidemic situation cannot be disclosed and shared in real time. These cause the inconvenience to grasp the health conditions of animals and plants. Thus, the correct measures and treatments are unable to be taken timely. Owing to the data information is not obtained timely and failure supervision, there is a health hazard that directly endangers the safety of food and survival of mankind health.

In the environment of coexistence and mutual benefit between human beings and plants, human beings have to master the growth status of plants in real time, monitor and ensure their healthy growth, which provides us with safe food, grains and fruits. It also provides a guarantee of healthy air for human beings.

In the environment of coexistence and mutual benefit between human beings and animals, human beings have to master the growth status of animals in real time, monitor and ensure their healthy growth, which provides us with safe food and meat. Protect us from being influenced by animal disasters and epidemics. It achieves the purpose of harmony coexistence on the Earth between human beings and animals.

People would install monitoring hardware equipment, such as camera equipment, recording equipment, on-site environmental monitoring equipment, hanging tags for the target things, smart monitoring settings and so on, in the required locations or places. The results generated from monitoring hardware are processed by single chip modules. Then it passes to the GPRS module, and the GPRS module transmits the video information to the remote management personnel through the wireless mobile network, so that the remote management personnel can grasp the activities and growth information of animals and plants in time. Through the big data, artificial intelligence (AI) and human wisdom, it supports the formulation of related protection policies.

Therefore, we have to find a new technical solution to solve the above difficulties.

### SUMMARY

All examples and features mentioned below may be combined in any technically possible way.

Implementations disclosed herein include a plant and animal health monitoring and management system and its method, using the big data technology to accomplish supervision networking, real-timing, safety, scientific, informatization and data sharing, and to realize an all-around supervision on the health conditions of plants and animals, that ensure a comprehensive, accurate and timely data collection, thus it improves job efficiency and productiveness. The technology of the present disclosure is suitable for plant and animal cultivation, breeding and transformation etc. and is widely appliable.

The present disclosure solves the above technical problems through the following technical solutions:

A plant and animal health monitoring management system, comprising a plant and animal health monitoring unit, an AI and artificial integrated health control unit, a big data monitoring management center unit, a plant and animal operation supervision unit, and a data analysis monitoring unit, wherein the AI and artificial integrated health control unit comprises an AI diagnosis and treatment control system, an AI treatment control system, an artificial intervention analysis system and an artificial intervention execution unit, the plant and animal operation supervision unit comprises a plant and animal administrator unit, a plant and animal owner unit, a plant and animal laboratory unit, a plant and animal research room unit, and a plant and animal integrated management unit, and a plant and animal enthusiast unit, the data analysis monitoring unit comprises an experimental research data analysis management unit, an integrated analysis management unit, and an on-site data analysis management unit, the plant and animal health monitoring unit is configured to monitor the health status of plants and animals to obtain health data information, and send the health data information to the AI and artificial integrated health control unit and the big data monitoring management center unit, the AI diagnosis and treatment control system is configured to set a health preset value, perform primary AI diagnosis and treatment control based on the health data information and the health preset value to obtain AI primary health diagnosis result information, send the AI primary health diagnosis result information and instruct the artificial intervention analysis system to perform primary artificial intervention analysis, receive primary artificial intervention analysis information of the artificial intervention analysis system and generate AI secondary health diagnosis result information, and send the AI secondary health diagnosis result information to the AI treatment control system, the AI treatment control system is configured to generate an AI treatment execution plan based on the AI secondary health diagnosis result information and send the AI treatment execution plan to the artificial intervention analysis system, the artificial intervention analysis system is configured to analyze the AI treatment execution plan and instruct the artificial intervention execution unit to perform artificial intervention, the artificial intervention execution unit is configured to perform artificial intervention and generate an artificial intervention execution report, the big data monitoring management center unit is configured to receive and integrate health data information sent by the plant and animal health monitoring unit, the AI diagnosis and treatment control system, the AI treatment control system, the artificial intervention analysis system, and the artificial intervention execution unit, respectively, the AI primary health diagnosis result information, the AI secondary health diagnosis result information, the AI treatment execution plan, and the artificial intervention result report, and is configured to generate a big data monitoring command;

the plant and animal administrator unit and the plant and animal owner unit are configured to perform on-site management according to the big data monitoring command, track and input on-site management information of the plants and animals, and send the on-site management information to the on-site data analysis management unit, the plant and animal laboratory unit and the plant and animal research room unit are configured to conduct experiments and researches on plants and animals respectively according to the big data monitoring command, track and input experimental research management information of the plants and animals, and send the experimental research management information to the experimental research data analysis management unit, the plant and animal integrated management unit is configured to integrate integrated management experience of the plants and animals, perform integrated management on the plants and animals according to the big data monitoring command to obtain integrated management information, and send the integrated management information to the integrated analysis management unit, and the on-site data analysis management unit is configured to receive experimental research analysis report of the experimental research data analysis management unit, receive integrated analysis report of the integrated analysis management unit, obtain an on-site data adjustment analysis report according to the experimental research analysis report and the integrated analysis report and feed them back to the plant and animal administrator unit and the plant and animal owner unit for on-site operation adjustment, and finally obtain on-site data information to feed the on-site data information back to the plant and animal health monitoring unit.

In some embodiments, the AI diagnosis and treatment control system comprises an AI diagnosis and treatment information analysis unit, a plant category or animal category selection unit, a large disease type database unit, an AI diagnosis and treatment disease type result unit, and an AI diagnosis and treatment plan result unit, the AI treatment control system comprises an AI treatment analysis unit, a large AI treatment database unit and an AI treatment plan result unit, and the artificial intervention analysis system comprises an artificial intervention analysis unit and an artificial treatment result unit; wherein

the plant category or animal category selection unit is configured to determine whether a subject of diagnosis and treatment of the AI diagnosis and treatment control system is a plant or an animal so as to obtain a category determination result, the large disease type database unit is configured to store and send disease type data to the AI diagnosis and treatment information analysis unit, the AI diagnosis and treatment information analysis unit is configured to perform diagnosis and treatment analysis according to the health data information, the on-site data information, the category determination result, and the disease type data so as to obtain first health diagnosis information, the AI diagnosis and treatment disease type result unit is configured to sort and analyze the first health diagnosis information to obtain the AI primary health diagnosis result information, and sends it to the artificial intervention analysis unit, the AI diagnosis and treatment information analysis unit is further configured to receive and analyze the artificial intervention analysis information of the artificial intervention analysis unit to obtain second health diagnosis information, and send it to the AI diagnosis and treatment plan result unit, the AI diagnosis and treatment plan result unit is configured to sort and analyze the second health diagnosis information to obtain the AI secondary health diagnosis information;

the large AI treatment database unit is used to store and send treatment data information to the AI treatment analysis unit, the AI treatment analysis unit is configured to obtain AI treatment analysis information according to the AI secondary health diagnosis information and the treatment data information, the AI diagnosis and treatment plan result unit is configured to obtain the AI treatment execution plan based on the analysis and sorting of the AI treatment analysis information, the artificial intervention analysis unit is configured to perform secondary artificial intervention analysis according to the AI treatment execution plan to obtain an artificial intervention execution plan, and the artificial treatment result unit is configured to analyze and sort the artificial intervention execution plan and obtain an artificial intervention execution report.

In some further embodiments, the big data monitoring management center unit comprises an AI diagnosis and treatment data module, an AI treatment data module, an artificial intervention analysis data module, an artificial intervention execution data module, a site condition data module, an on-site monitoring data module, and an industry information intelligence data module.

In some embodiments, the plant and animal health monitoring management system comprises a data interface unit which is provided between the big data monitoring management center unit and the plant and animal operation supervision unit.

In some embodiments, the plant and animal operation supervision unit comprises the plant and animal enthusiast unit which is configured to perform breeding observation of plants and animals based on the big data monitoring command.

In some embodiments, the data analysis monitoring unit comprises an industry help module, and/or an industry supervision module, and/or an industry intelligence module, and/or a business guidance module, wherein the industry help module, the industry supervision module, the industry intelligence module, and the business guidance modules are all connected to the integrated analysis management unit.

In some embodiments, the plant and animal health monitoring unit comprises a plant and animal environmental sunray monitoring module, a plant and animal environmental temperature monitoring module, a plant and animal environmental humidity monitoring module, a plant and animal environmental air monitoring module, a plant body environmental soil monitoring module, and a plant body plant attachment monitoring module, a plant body animal attachment monitoring module, a plant body nutrition monitoring module, and an animal body food monitoring module; the artificial intervention execution unit correspondingly comprises a plant and animal artificial intervention environmental sunray module, a plant and animal artificial intervention environmental temperature module, a plant and animal artificial intervention environmental humidity module, a plant and animal artificial intervention environmental air module, a plant body artificial intervention environmental soil module, a plant body plant attachment artificial intervention module, a plant body animal attachment artificial intervention module, a plant body nutrition artificial intervention module, and an animal body food artificial intervention module.

In some embodiments, the artificial intervention execution unit comprises a plant body medicine treatment module, and/or an animal body medicine treatment module, and/or a plant body safety isolation treatment module, and/or an animal body safety isolation treatment module, and/or a plant body safety killing module, and/or an animal body safety killing module, the plant and animal health monitoring unit further comprises an animal body temperature monitoring module, an animal body heart rate monitoring module, an animal body liver, gallbladder, stomach, spleen, lung monitoring module, an animal body excretion and urine monitoring module, and/or a plant and animal real-time audio and video monitoring module, the plant and animal environmental sunray monitoring module, the plant and animal environmental temperature monitoring module, the plant and animal environmental humidity monitoring module, the plant and animal environmental air monitoring module, the plant body environmental soil monitoring module, the plant body plant attachment monitoring module, the plant body animal attachment monitoring module, the plant body nutrition monitoring module, and the plant and animal real-time audio and video monitoring module are all correspondingly artificially interfered by the plant body medicine treatment module, and/or the plant body safety isolation treatment module, and/or the plant body safety killing module; and the plant and animal environmental sunray monitoring module, the plant and animal environmental temperature monitoring module, the plant and animal environmental humidity monitoring module, the plant and animal environmental air monitoring module, the animal body food monitoring module, the animal body temperature monitoring module, the animal body heart rate monitoring module, the animal body liver, gallbladder, stomach, spleen, lung monitoring module, an animal body excretion and urine monitoring module, and the plant and animal real-time audio and video monitoring module are all correspondingly artificially interfered by the animal body medicine treatment module, and/or the animal body safety isolation treatment module, and/or the animal body safety killing module.

A plant and animal health monitoring management system, comprising the following steps:
step S1, performing health monitoring on plants and animals to obtain health data information;
step S2, performing primary AI diagnosis and treatment control to obtain AI primary health diagnosis result information;
step S3, performing primary artificial intervention analysis to obtain primary artificial intervention analysis information;
step S4, performing secondary AI diagnosis and treatment control to obtain AI secondary health diagnosis result information;
step S5, performing AI treatment control to obtain an AI treatment execution plan;
step S6, performing secondary artificial intervention analysis to obtain an artificial intervention execution plan;
step S7, performing artificial intervention;
step S8, performing big data monitoring management;
step S9, performing operation supervision on plants and animals, comprising:
step S9A, performing on-site supervision to obtain on-site management information; step S9B, performing experimental research supervision to obtain experimental research management information; and step S9C, performing integrated supervision to obtain integrated management information; and
step S10, performing data analysis and monitoring, comprising: step S10A, analyzing and monitoring, after the step S9A, the on-site management information to obtain an on-site data adjustment analysis report; step S10B, analyzing and monitoring, after the step S9B, the experimental research management information to obtain an experimental research analysis report; and step S10C, analyzing and monitoring, after the step S9C, the integrated management information to obtain an integrated analysis report, wherein:

the experimental research analysis report obtained in step S10B and the integrated analysis report obtained in step S10C both enter step S10A, and on-site data information is obtained through step S10A, and the on-site data information is fed back to step S2.

### BRIEF DESCRIPTION OF THE DRAWINGS

These and other features of the present embodiments will be understood better by reading the following detailed description, taken together with the figures herein described. In the drawings, each identical or nearly identical component that is illustrated in various figures may be represented by a like numeral. For purposes of clarity, not every component may be labeled in every drawing.

FIG. 1 is a schematic diagram of the overall structure of the plant and animal health monitoring management system, in accordance with some embodiments of the present disclosure.

FIG. 2 is a structure diagram of the AI and artificial integrated health control unit, in accordance with some embodiments of the present disclosure.

FIG. 3 is a structure diagram of the plant and animal health monitoring unit and the artificial intervention execution unit, in accordance with some embodiments of the present disclosure.

FIG. 4 is a structure diagram of the big data monitoring center unit, in accordance with some embodiments of the present disclosure.

FIG. 5 is a structure diagram of plant and animal health monitoring management method, in accordance with some embodiments of the present disclosure.

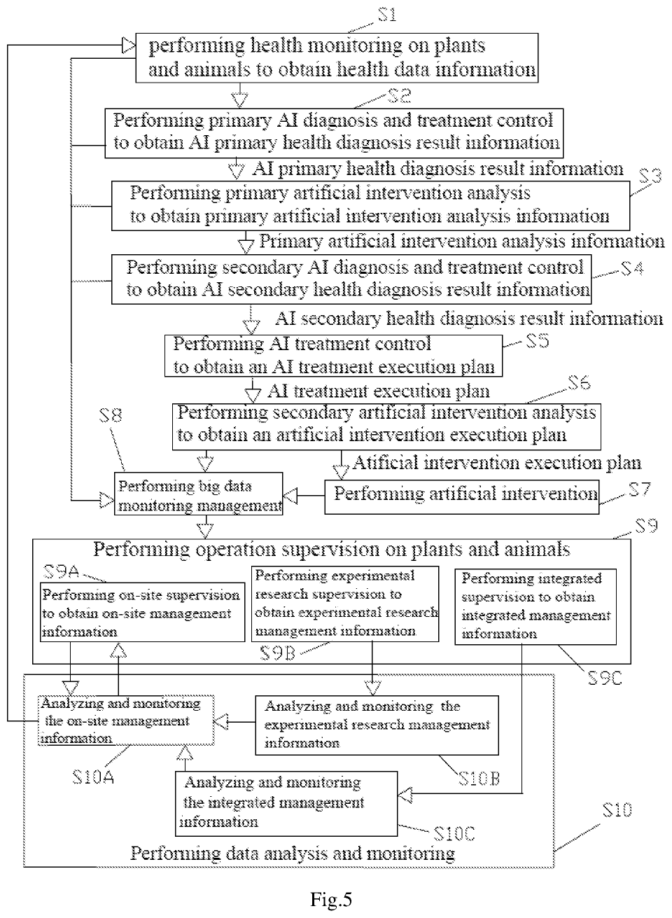

### DETAILED DESCRIPTION OF THE DISCLOSURE

The specific embodiments of the disclosure will be further described in combination with the appended drawings and embodiments. The following embodiments are only used to explain the technical solution of the disclosure more clearly and cannot limit the protection scope of the disclosure.

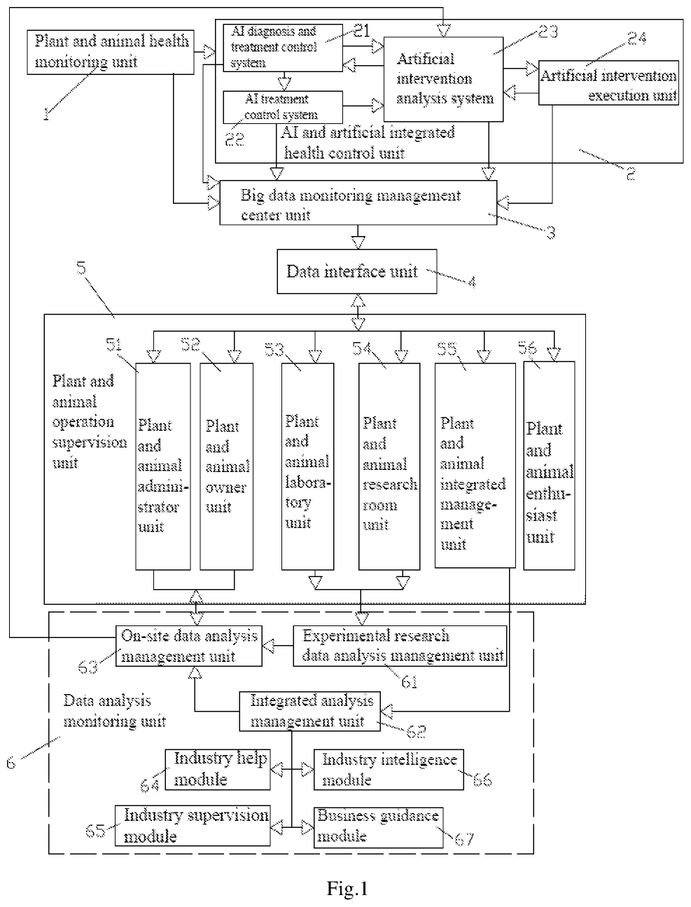

As shown in FIG. 1, this disclosure includes a plant and animal health monitoring management system, comprising a plant and animal health monitoring unit 1, an AI and artificial integrated health control unit 2, a big data monitoring management center unit 3, a plant and animal operation supervision unit 5, and a data analysis monitoring unit 6, wherein the AI and artificial integrated health control unit 2 comprises an AI diagnosis and treatment control system 21, an AI treatment control system 22, an artificial intervention analysis system 23 and an artificial intervention execution unit 24. The plant and animal operation supervision unit 5 comprises a plant and animal administrator unit 51, a plant and animal owner unit 52, a plant and animal laboratory unit 53, a plant and animal research room unit 54, a plant and animal integrated management unit 55 and a plant and animal enthusiast unit 56. The data analysis monitoring unit 6 comprises an experimental research data analysis management unit 61, an integrated analysis management unit 62, and an on-site data analysis management unit 63. The plant and animal health monitoring unit 1 is configured to monitor the health status of plants and animals to obtain health data information, and send the health data information to the AI and artificial integrated health control unit 2 and a big data monitoring management center unit 3, the AI diagnosis and treatment control system 21 is configured to set a health preset value, perform primary AI diagnosis and treatment control based on the health data information and the health preset value to obtain AI primary health diagnosis result information, send the AI primary health diagnosis result information and instruct the artificial intervention analysis system 23 to perform primary artificial intervention analysis, receive primary artificial intervention analysis information of the artificial intervention analysis system 23 and generate AI secondary health diagnosis result information, and send the AI secondary health diagnosis result information to the AI treatment control system 22, the AI treatment control system 22 is configured to generate an AI treatment execution plan based on the AI secondary health diagnosis result information and send the AI treatment execution plan to the artificial intervention analysis system 23, the artificial intervention analysis system 23 is configured to analyze the AI treatment execution plan and instruct the artificial intervention execution unit 24 to perform artificial intervention, the artificial intervention execution unit 24 is configured to perform artificial intervention and generate an artificial intervention execution report, a big data monitoring management center unit 3 is configured to receive and integrate health data information sent by the plant and animal health monitoring unit, the AI diagnosis and treatment control system 21, the AI treatment control system 22, the artificial intervention analysis system 23, and the artificial intervention execution unit 24 respectively, the AI primary health diagnosis result information, the AI secondary health diagnosis result information, the AI treatment execution plan, and the artificial intervention result report, and is configured to generate a big data monitoring command.

The plant and animal administrator unit 51 and the plant and animal owner unit 52 are configured to perform on-site management according to the big data monitoring command, track and input on-site management information of the plants and animals, and send the on-site management information to the on-site data analysis management unit 63, the plant and animal laboratory unit 53 and the plant and animal research room unit 54 are configured to conduct experiments and researches on plants and animals respectively according to the big data monitoring command, track and input experimental research management information of the plants and animals, and send the experimental research management information to the experimental research data analysis management unit 61, the plant and animal integrated management unit is configured to integrate integrated management experience of the plants and animals, perform integrated management on the plants and animals according to the big data monitoring command to obtain integrated management information, and send the integrated management information to the integrated analysis management unit 62, and the on-site data analysis management unit 63 is configured to receive experimental research analysis report of the experimental research data analysis management unit 61, receive integrated analysis report of the integrated analysis management unit 62, obtain an on-site data adjustment analysis report according to the experimental research analysis report and the integrated analysis report and feed them back to the plant and animal administrator unit 51 and the plant and animal owner unit 52 for on-site operation adjustment, and finally obtain on-site data information to feed the on-site data information back to the plant and animal health monitoring unit 1.

The present disclosure uses a big data technology to realize supervision networking, real-timing, safety, informatization and data sharing, carry out an all-round effective supervision and control on the health conditions of plants and animals, manage to fulfill a comprehensive supervision, accurate and real-time monitoring on plants and animals, to ensure the health control of plants and animals are being effective, safe, accurate, scientific, real-time and enhance job efficiency and productiveness.

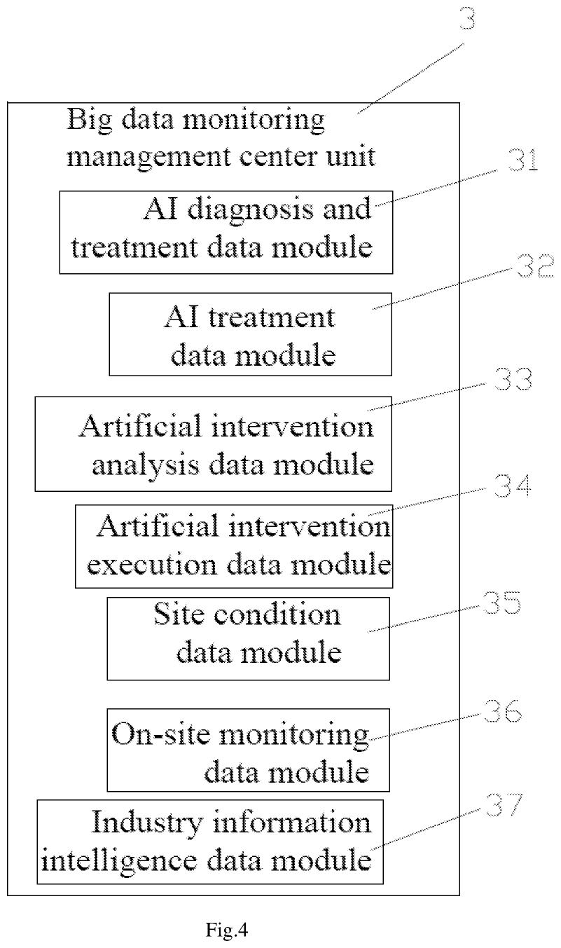

As shown in FIG. 4, this disclosure includes the big data monitoring management center unit 3 comprises an AI diagnosis and treatment data module 31, an AI treatment data module 32, an artificial intervention analysis data module 33, an artificial intervention execution data module 34, a site condition data module 35, an on-site monitoring data module 36, and an industry information intelligence data module 37. An AI diagnosis and treatment data module 31, an AI treatment data module 32, an artificial intervention analysis data module 33, an artificial intervention execution data module 34 receive the data information from AI diagnosis control system 21, AI treatment control system 22, artificial intervention analysis system 23 and artificial intervention execution unit 24 respectively. A site condition data module 35, an on-site monitoring data module 36 and an industry information intelligence data module 37 etc receive the data information from the plant and animal health monitoring unit 1, wherein the plant and animal health monitoring unit 1 collects and integrates the data information through the on-site data analysis management unit 63 and analyzes the data information altogether from the various aspects.

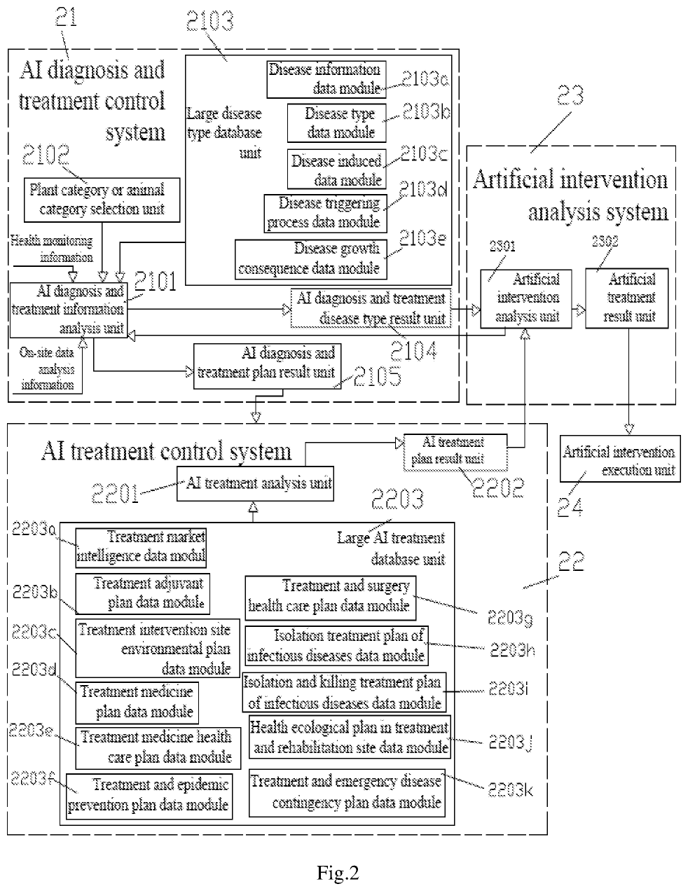

As shown in FIG. 2, the AI diagnosis and treatment control system 21 comprises an AI diagnosis and treatment information analysis unit 2101, a plant category or animal category selection unit 2102, a large disease type database unit 2103, an AI diagnosis and treatment disease type result unit 2104, and an AI diagnosis and treatment plan result unit 2105. The large disease type database unit 2103 comprises disease information data module 2103a, a disease type data module 2103b, a disease induced data module 2103c, a disease triggering process data module 2103d and a disease growth consequence data module 2103e, the AI treatment control system 22 comprises of AI treatment analysis unit 2201, large AI treatment database unit 2203 and AI treatment plan result unit 2202, the artificial intervention analysis system 23 comprises of artificial intervention analysis unit 2301 and artificial treatment result unit 2302; wherein:

the plant category or animal category selection unit is configured to determine whether a subject of diagnosis and treatment of the AI diagnosis and treatment control system 21 is a plant or an animal so as to obtain a category determination result, the large disease type database unit 2103 is configured to store and send disease type data to the AI diagnosis and treatment information analysis unit 2101, the AI diagnosis and treatment information analysis unit 2101 is configured to perform diagnosis and treatment analysis according to the health data information, the on-site data information, the category determination result, and the disease type data so as to obtain first health diagnosis information, the AI diagnosis and treatment disease type result unit 2104 is configured to sort and analyze the first health diagnosis information to obtain the AI primary health diagnosis result information, and sends it to the artificial intervention analysis unit 2301, the AI diagnosis and treatment information analysis unit 2101 is further configured to receive and analyze the artificial intervention analysis information of the artificial intervention analysis unit 2301 to obtain second health diagnosis information, and send it to the AI diagnosis and treatment plan result unit 2105, the AI diagnosis and treatment plan result unit 2105 is configured to sort and analyze the second health diagnosis information to obtain the AI secondary health diagnosis information;

the large AI treatment database unit 2203 is used to store and send treatment data information to the AI treatment analysis unit 2201, the large AI treatment database unit 2203 comprises a treatment market intelligence data module 2203a, a treatment adjuvant plan data module 2203b, a treatment intervention site environmental plan data module 2203c, a treatment medicine plan data module 2203d, a treatment medicine health care plan data module 2203e, a treatment and epidemic prevention plan data module 2203f, a treatment and surgery health care plan data module 2203g, an isolation treatment plan of infectious diseases data module 2203h, an isolation and killing treatment plan of infectious diseases data module 2203i, a health ecological plan in treatment and rehabilitation site data module 2203j, and a treatment and emergency disease contingency plan data module 2203k etc.

The treatment market intelligence data module 2203a provides global reference information for the target bodies, including real-time epidemic disease and epidemic situation, real-time diagnosis and treatment plan, real-time human intervention result, global impact of the disease and its epidemic, it is to announce an alert and provide a prevention action to other people.

The treatment adjuvant plan data module 2203b provides an adjuvant treatment plan for the target bodies. The improvement measures come out including real-time monitor on the disease and epidemic situation of the target bodies in other regions, such as stricter cleaning measures for the target bodies than usual, stricter disinfection and cleaning measures on the site etc.

The treatment intervention site environmental plan data module 2203c is to intervene the site environment according to the health status of the target bodies. For example: on-site weeding and insects control, disinfecting and detoxification, improving sunlight and lighting, improving air circulation, improving on-site constant temperature environment.

The treatment medicine plan data module 2203d is to make a medication treatment based on the disease or epidemic situation of the target bodies. For example: disinfectants, anti-inflammatory drugs, cold drugs and any medicine for the target bodies.

The treatment medicine health care plan data module 2203e is to plan a health care program based on the side effects after drugs taken, it is because the target bodies would suffer from a certain level of side effects as long as drugs are in use. After drugs are taken to achieve its purpose, the target bodies have to proceeded care maintenance measures, such as: improve the balance of qi and blood in the bodies, get additional nutrients etc.

The treatment and epidemic prevention plan data module 2203f is to take corresponding preventive measures based on the real-time update on disease and epidemic situation of the target bodies around the world, including real-time isolation from contacting external stuffs, such as: water, food, moving people, moving animals. To get rid of infection, strict disinfection and anti-virus programs are adopted whenever external objects contact the target bodies.

The treatment and surgery health care plan data module 2203g is to do surgical treatment on the target bodies. When drug is not effective or it is not suitable for medicine, surgery is adoptable for treatment and with post-surgery maintenance measures.

The isolation treatment plan of infectious diseases data module 2203h is to trigger a timely isolation measure on the situation that infectious disease and epidemic situation threaten the target bodies, it prevents infecting other target bodies and expose to a greater risk. For example: independent isolation feeding, disinfection, medication, surgery, maintenance etc.

The isolation and killing treatment plan of infectious diseases data module 2203i is the action after thorough evaluation on the target bodies which has been gone through the rescue. In case the condition does not get improvement or even worser within a certain period of time, immediate isolation and pollution-free killing measures will be taken in order to reduce or eliminate the risk of infection.

The health ecological plan in treatment and rehabilitation site data module 2203j is for site ecological rehabilitation after occurrence of disease or epidemic situation, including disinfection of water sources, food, on-site facilities and the environment. It is to restore the ecology rehabilitation of the site. Perform scientific, quantitative and reasonable data processing for each task. To ensure that there is no impact on the breeding or cultivation of next batch of the target bodies.

The treatment and emergency disease contingency plan data module 2203k is the emergency measures for the target bodies, including setting up of isolation rooms, medication rooms, surgery rooms, on-site first-level cordon, second-level cordon, any ad hoc emergency rehearsal and measures, and reporting infectious diseases to supervision department and vigilance work. Rehearsal and training are available for the working staff. The working staff need to be well-trained and assessed by the relevant regulatory authorities about health caring of target bodies, as well as its rescue, medication, and surgery. Only when staff get passed the assessment, they are certified to work.

The AI treatment analysis unit 2201 is configured to obtain the AI treatment analysis information according to the AI secondary health diagnosis information and the treatment data information, the AI diagnosis and treatment plan result unit 2105 is configured to obtain the AI treatment execution plan based on the analysis and sorting of the AI treatment analysis information, the artificial intervention analysis unit is configured to perform secondary artificial intervention analysis according to the AI treatment execution plan to obtain an artificial intervention execution plan, and the artificial treatment result unit is configured to analyze and sort the artificial intervention execution plan and obtain an artificial intervention execution report.

In an embodiment, the plant and animal health monitoring management systems comprises a data interface unit 4, which is provided between the big data monitoring management center unit 3 and the plant and animal operation supervision unit 5.

In other embodiment, the plant and animal operation supervision unit 5 comprises the plant and animal enthusiast unit 56, which is configured to perform breeding observation of plants and animals based on the big data monitoring command.

As shown in FIG. 1, the data analysis monitoring unit 6 comprises an industry help module 64, and/or an industry supervision module 65, and/or an industry intelligence module 66, and/or a business guidance module 67, wherein the industry help module 64, the industry supervision module 65, the industry intelligence module 66, and the business guidance module 67 are all connected to the integrated analysis management unit 62. The industry help module 64 is the industry supervision and management department that subsidize with goods, medicine, funding and any supportive compensation for operators who are suffering difficulties in running the business.

The industry supervision module 65 is the industry supervision and management department that conduct necessary monitoring to the operators or their premises, provide training and learning courses to working staffs or supervisors, ensure each personnel are assessed and qualified before work. The industry intelligence module 66 is the industry supervision and management department that provide real-time and up-to-date intelligence to the operators or their premises from global industry as an operation guidance reference. The business guidance module 67 is an industry supervision and management department that provide scientific, reasonable, smart, safe and health guidance to the operators and their premises, so that operators or practitioner can monitor the health condition of the target bodies effectively.

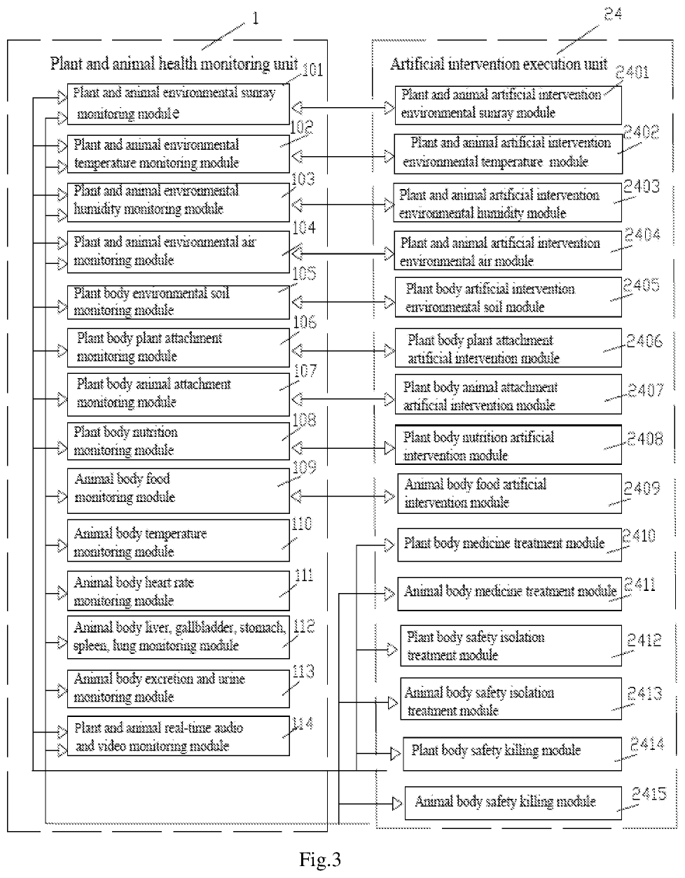

In another embodiment, as shown in FIG. 3, the plant and animal health monitoring unit 1 comprises a plant and animal environmental sunray monitoring module 101, a plant and animal environmental temperature monitoring module 102, a plant and animal environmental humidity monitoring module 103, a plant and animal environmental air monitoring module 104, a plant body environmental soil monitoring module 105, a plant body plant attachment monitoring module 106, a plant body animal attachment monitoring module 107, a plant body nutrition monitoring module 108, and an animal body food monitoring module 109; the artificial intervention execution unit 24 are correspondingly comprises of a plant and animal artificial intervention environmental sunray module 2401, a plant and animal artificial intervention environmental temperature monitoring module 2402, a plant and animal artificial intervention environmental humidity module 2403, a plant and animal artificial intervention environmental air module 2404, a plant body artificial intervention environmental soil module 2405, a plant body plant attachment artificial intervention module 2406, a plant body animal attachment artificial intervention module 2407, a plant body nutrition artificial intervention module 2408, and an animal body food artificial intervention module 2409.

In some embodiments, the artificial intervention execution unit 24 comprises a plant body medicine treatment module 2410, and/or an animal body medicine treatment module 2411, and/or a plant body safety isolation treatment module 2412, and/or an animal body safety isolation treatment module 2413, and/or a plant body safety killing module 2414, and/or an animal body safety killing module 2415, the plant and animal health monitoring unit 1 further comprises an animal body temperature monitoring module 110, an animal body heart rate monitoring module 111, an animal body liver, gallbladder, stomach, spleen, lung monitoring module 112, an animal body excretion and urine monitoring module 113, and/or a plant and animal real-time audio and video monitoring module 114, the plant and animal environmental sunray monitoring module 101, the plant and animal environmental temperature monitoring module 102, the plant and animal environmental humidity monitoring module 103, the plant and animal environmental air monitoring module 104, the plant body environmental soil monitoring module 105, the plant body plant attachment monitoring module 106, the plant body animal attachment monitoring module 107, the plant body nutrition monitoring module 108, and the plant and animal real-time audio and video monitoring module 114 are all correspondingly artificially interfered by the plant body medicine treatment module 2410, and/or the plant body safety isolation treatment module 2412, and/or the plant body safety killing module 2414; and the plant and animal environmental sunray monitoring module 101, the plant and animal environmental temperature monitoring module 102, the plant and animal environmental humidity monitoring module 103, the plant and animal environmental air monitoring module 104, the animal body food monitoring module 109, the animal body temperature monitoring module 110, the animal body heart rate monitoring module 111, the animal body liver, gallbladder, stomach, spleen, lung monitoring module 112, an animal body excretion and urine monitoring module 113 and the plant and animal real-time audio and video monitoring module 114 are all correspondingly artificially interfered by the animal body medicine treatment module 2411, and/or the animal body safety isolation treatment module 2412, and/or the animal body safety killing module 2415.

A plant and animal health monitoring management system, comprising the following steps:
step S1, performing health monitoring on plants and animals to obtain health data information;
step S2, performing primary AI diagnosis and treatment control to obtain AI primary health diagnosis result information;
step S3, performing primary artificial intervention analysis to obtain primary artificial intervention analysis information;
step S4, performing secondary AI diagnosis and treatment control to obtain AI secondary health diagnosis result information;
step S5, performing AI treatment control to obtain an AI treatment execution plan;
step S6, performing secondary artificial intervention analysis to obtain an artificial intervention execution plan;
step S7, performing artificial intervention;
step S8, performing big data monitoring management;
step S9, performing operation supervision on plants and animals, comprising:
step S9A, performing on-site supervision to obtain on-site management information; step S9B, performing experimental research supervision to obtain experimental research management information; and step S9C, performing integrated supervision to obtain integrated management information; and
step S10, performing data analysis and monitoring, comprising: step S10A, analyzing and monitoring, after the step S9A, the on-site management information to obtain an on-site data adjustment analysis report; step S10B, analyzing and monitoring, after the step S9B, the experimental research management information to obtain an experimental research analysis report; and step S10C, analyzing and monitoring, after the step S9C, the integrated management information to obtain an integrated analysis report, wherein:

the experimental research analysis report obtained in step S10B and the integrated analysis report obtained in step S10C both enter step S10A, and on-site data information is obtained through step S TOA, and the on-site data information is fed back to step S2.

The present disclosure uses a big data technology to realize supervision networking, real-timing, safety, informatization and data sharing, carry out an all-round effective supervision and control on the health conditions of plants and animals, manage to fulfill a comprehensive supervision, accurate and real-time monitoring on plants and animals, to ensure the health control of plants and animals are being effective, safe, accurate, scientific, real-time, and enhance job efficiency and productiveness. The disclosure realizes the technological, informatization, safety, and intelligent cultivation of plants, captive breeding and transformation of animals. It achieves global information sharing, resource sharing, cooperative, meet the desire of cooperation, mutually beneficial, sharing and coexistence among human beings, plants and animal on the Earth. This disclosure can be applicable widely in use.

Although the methods and systems have been described relative to specific embodiments thereof, they are not so limited. Many modifications and variations may become apparent in light of the above teachings. Many additional changes in the details, materials, and arrangement of parts, herein described and illustrated, may be made by those skilled in the art. A number of implementations have been described. Nevertheless, it will be understood that additional modifications may be made without departing from the scope of the inventive concepts described herein, and, accordingly, other implementations are within the scope of the following claims.

What is claimed is:

1. A plant and animal health monitoring management system, comprising a plant and animal health monitoring unit, an AI and artificial integrated health control unit, a big data monitoring management center unit, a plant and animal operation supervision unit, and a data analysis monitoring unit, wherein the AI and artificial integrated health control unit comprises an AI diagnosis and treatment control system, an AI treatment control system, an artificial intervention analysis system and an artificial intervention execution unit, the plant and animal operation supervision unit comprises a plant and animal administrator unit, a plant and animal owner unit, a plant and animal laboratory unit, a plant and animal research room unit, and a plant and animal integrated management unit, and a plant and animal enthusiast unit, the data analysis monitoring unit comprises an experimental research data analysis management unit, an integrated analysis management unit, and an on-site data analysis management unit, the plant and animal health monitoring unit is configured to monitor the health status of plants and animals to obtain health data information, and send the health data information to the AI and artificial integrated health control unit and the big data monitoring management center unit, the AI diagnosis and treatment control system is configured to set a health preset value, perform primary AI diagnosis and treatment control based on the health data information and the health preset value to obtain AI primary health diagnosis result information, send the AI primary health diagnosis result information and instruct the artificial intervention analysis system to perform primary artificial intervention analysis, receive primary artificial intervention analysis information of the artificial intervention analysis system and generate AI secondary health diagnosis result information, and send the AI secondary health diagnosis result information to the AI treatment control system, the AI treatment control system is configured to generate an AI treatment execution plan based on the AI secondary health diagnosis result information and send the AI treatment execution plan to the artificial intervention analysis system, the artificial intervention analysis system is configured to analyze the AI treatment execution plan and instruct the artificial intervention execution unit to perform artificial intervention, the artificial intervention execution unit is configured to perform artificial intervention and generate an artificial intervention execution report, the big data monitoring management center unit is configured to receive and integrate health data information sent by the plant and animal health monitoring unit, the AI diagnosis and treatment control system, the AI treatment control system, the artificial intervention analysis system, and the artificial intervention execution unit, respectively, the AI primary health diagnosis result information, the AI secondary health diagnosis result information, the AI treatment execution plan, and the artificial intervention result report, and is configured to generate a big data monitoring command;
the plant and animal administrator unit and the plant and animal owner unit are configured to perform on-site management according to the big data monitoring command, track and input on-site management information of the plants and animals, and send the on-site management information to the on-site data analysis management unit, the plant and animal laboratory unit and the plant and animal research room unit are configured to conduct experiments and researches on plants and animals respectively according to the big data monitoring command, track and input experimental research management information of the plants and animals, and send the experimental research management information to the experimental research data analysis management unit, the plant and animal integrated management unit is configured to integrate integrated management experience of the plants and animals, perform integrated management on the plants and animals according to the big data monitoring command to obtain integrated management information, and send the integrated management information to the integrated analysis management unit, and the on-site data analysis management unit is configured to receive experimental research analysis report of the experimental research data analysis management unit, receive integrated analysis report of the integrated analysis management unit, obtain an on-site data adjustment analysis report according to the experimental research analysis report and the integrated analysis report and feed them back to the plant and animal administrator unit and the plant and animal owner unit for on-site operation adjustment, and finally obtain on-site data information to feed the on-site data information back to the plant and animal health monitoring unit.

2. The plant and animal health monitoring management system according to claim 1, wherein the AI diagnosis and treatment control system comprises an AI diagnosis and treatment information analysis unit, a plant category or animal category selection unit, a large disease type database unit, an AI diagnosis and treatment disease type result unit, and an AI diagnosis and treatment plan result unit, the AI treatment control system comprises an AI treatment analysis unit, a large AI treatment database unit and an AI treatment plan result unit, and the artificial intervention analysis system comprises an artificial intervention analysis unit and an artificial treatment result unit; wherein
the plant category or animal category selection unit is configured to determine whether a subject of diagnosis and treatment of the AI diagnosis and treatment control system is a plant or an animal so as to obtain a category determination result, the large disease type database unit is configured to store and send disease type data to the AI diagnosis and treatment information analysis unit, the AI diagnosis and treatment information analysis unit is configured to perform diagnosis and treatment analysis according to the health data information, the on-site data information, the category determination result, and the disease type data so as to obtain first health diagnosis information, the AI diagnosis and treatment disease type result unit is configured to sort and analyze the first health diagnosis information to obtain the AI primary health diagnosis result information, and sends it to the artificial intervention analysis unit, the AI diagnosis and treatment information analysis unit is further configured to receive and analyze the artificial intervention analysis information of the artificial intervention analysis unit to obtain second health diagnosis information, and send it to the AI diagnosis and treatment plan result unit, the AI diagnosis and treatment plan result unit is configured to sort and analyze the second health diagnosis information to obtain the AI secondary health diagnosis information;
the large AI treatment database unit is used to store and send treatment data information to the AI treatment analysis unit, the AI treatment analysis unit is configured to obtain AI treatment analysis information according to the AI secondary health diagnosis information and the treatment data information, the AI diagnosis and treatment plan result unit is configured to obtain the AI treatment execution plan based on the analysis and sorting of the AI treatment analysis information, the artificial intervention analysis unit is configured to perform secondary artificial intervention analysis according to the AI treatment execution plan to obtain an artificial intervention execution plan, and the artificial treatment result unit is configured to analyze and sort the artificial intervention execution plan and obtain an artificial intervention execution report.

3. The plant and animal health monitoring management system according to claim 1, wherein the big data monitoring management center unit comprises an AI diagnosis and treatment data module, an AI treatment data module, an artificial intervention analysis data module, an artificial intervention execution data module, a site condition data module, an on-site monitoring data module, and an industry information intelligence data module.

4. The plant and animal health monitoring management system according to claim 1, wherein the plant and animal health monitoring management system comprises a data interface unit which is provided between the big data monitoring management center unit and the plant and animal operation supervision unit.

5. The plant and animal health monitoring management system according to claim 1, wherein the plant and animal operation supervision unit comprises the plant and animal enthusiast unit which is configured to perform breeding observation of plants and animals based on the big data monitoring command.

6. The plant and animal health monitoring management system according to claim 1, wherein the data analysis monitoring unit comprises an industry help module, and/or an industry supervision module, and/or an industry intelligence module, and/or a business guidance module, wherein the industry help module, the industry supervision module, the industry intelligence module, and the business guidance modules are all connected to the integrated analysis management unit.

7. The plant and animal health monitoring management system according to claim 1, wherein the plant and animal health monitoring unit comprises a plant and animal environmental sunray monitoring module, a plant and animal environmental temperature monitoring module, a plant and animal environmental humidity monitoring module, a plant and animal environmental air monitoring module, a plant body environmental soil monitoring module, and a plant body plant attachment monitoring module, a plant body animal attachment monitoring module, a plant body nutrition monitoring module, and an animal body food monitoring module; the artificial intervention execution unit correspondingly comprises a plant and animal artificial intervention environmental sunray module, a plant and animal artificial intervention environmental temperature module, a plant and animal artificial intervention environmental humidity module, a plant and animal artificial intervention environmental air module, a plant body artificial intervention environmental soil module, a plant body plant attachment artificial intervention module, a plant body animal attachment artificial intervention module, a plant body nutrition artificial intervention module, and an animal body food artificial intervention module.

8. The plant and animal health monitoring management system according to claim 7, wherein the artificial intervention execution unit comprises a plant body medicine treatment module, and/or an animal body medicine treatment module, and/or a plant body safety isolation treatment module, and/or an animal body safety isolation treatment module, and/or a plant body safety killing module, and/or an animal body safety killing module, the plant and animal health monitoring unit further comprises an animal body temperature monitoring module, an animal body heart rate monitoring module, an animal body liver, gallbladder, stomach, spleen, lung monitoring module, an animal body excretion and urine monitoring module, and/or a plant and animal real-time audio and video monitoring module, the plant and animal environmental sunray monitoring module, the plant and animal environmental temperature monitoring module, the plant and animal environmental humidity monitoring module, the plant and animal environmental air monitoring module, the plant body environmental soil monitoring module, the plant body plant attachment monitoring module, the plant body animal attachment monitoring module, the plant body nutrition monitoring module, and the plant and animal real-time audio and video monitoring module are all correspondingly artificially interfered by the plant body medicine treatment module, and/or the plant body safety isolation treatment module, and/or the plant body safety killing module; and the plant and animal environmental sunray monitoring module, the plant and animal environmental temperature monitoring module, the plant and animal environmental humidity monitoring module, the plant and animal environmental air monitoring module, the animal body food monitoring module, the animal body temperature monitoring module, the animal body heart rate monitoring module, the animal body liver, gallbladder, stomach, spleen, lung monitoring module, and the plant and animal real-time audio and video monitoring module are all correspondingly artificially interfered by the animal body medicine treatment module, and/or the animal body safety isolation treatment module, and/or the animal body safety killing module.

9. A plant and animal health monitoring management system, comprising the following steps:
step S1, performing health monitoring on plants and animals to obtain health data information;
step S2, performing primary AI diagnosis and treatment control to obtain AI primary health diagnosis result information;
step S3, performing primary artificial intervention analysis to obtain primary artificial intervention analysis information;
step S4, performing secondary AI diagnosis and treatment control to obtain AI secondary health diagnosis result information;
step S5, performing AI treatment control to obtain an AI treatment execution plan;
step S6, performing secondary artificial intervention analysis to obtain an artificial intervention execution plan;
step S7, performing artificial intervention;
step S8, performing big data monitoring management;
step S9, performing operation supervision on plants and animals, comprising:
step S9A, performing on-site supervision to obtain on-site management information; step S9B, performing experimental research supervision to obtain experimental research management information; and step S9C, performing integrated supervision to obtain integrated management information; and
step S10, performing data analysis and monitoring, comprising: step S10A, analyzing and monitoring, after the step S9A, the on-site management information to obtain an on-site data adjustment analysis report; step S10B, analyzing and monitoring, after the step S9B, the experimental research management information to obtain an experimental research analysis report; and step S10C, analyzing and monitoring, after the step S9C, the integrated management information to obtain an integrated analysis report, wherein:
the experimental research analysis report obtained in step S10B and the integrated analysis report obtained in step S10C both enter step S10A, and on-site data information is obtained through step S10A, and the on-site data information is fed back to step S2.
